(19) United States
(12) Patent Application Publication
(10) Pub. No.: US 2010/0152213 A1
(43) Pub. Date: Jun. 17, 2010

(54) COMPOUND THAT IS A DUAL INHIBITOR OF ENZYMES PDE7 AND/OR PDE4, PHARMACEUTICAL COMPOSITIONS AND USES THEREOF

(76) Inventors: Carmen Gil Ayuso-Gontan, Madrid (ES); Tania Castaño Calduch, Madrid (ES); Nuria Campillo Martin, Madrid (ES); Sara Ballester Jareño, Madrid (ES); Carol González Garcia, Madrid (ES); Francisco Javier Hernández Torres, Madrid (ES)

Correspondence Address: IP Patent Docketing;K&L GATES LLP, 599 Lexington Avenue, 33rd Floor, New York, NY, 10022-6030, US

(21) Appl. No.: 12/532,104

(22) PCT Filed: Mar. 14, 2008
(86) PCT No.: PCT/ES2008/070051

§ 371 Date: Jan. 7, 2010

(30) Foreign Application Priority Data
Mar. 22, 2007 (ES) P 200700762

(51) Int. Cl. A61K 31/517; A61P 37/06

(52) U.S. Cl. 514/266.3

(57) ABSTRACT

The invention relates to a series of dual inhibitors of enzymes PDE7 and PDE4, having formula (I) and to the use thereof in the production of pharmaceutical compositions for the treatment of inflammatory and/or autoimmune processes.

### CROSS REFERENCE TO RELATED APPLICATIONS AND PRIORITY CLAIM

This application claims the benefit under 35 U.S.C. §371 to PCT/ES2008/070051 filed Mar. 14, 2008, which claims the benefit of Patent Application P 200700762 filed Mar. 22, 2007 in Spain. The entire disclosures of both applications are incorporated by reference herein.

### BACKGROUND

1. Technical Field

The present disclosure is aimed at the field of medical chemistry, more specifically at dual inhibitors of enzymes PDE7 and PDE4 and their potential use in the preparation of pharmaceutical compositions for the treatment of inflammatory or autoimmune processes; and therefore, it relates to the pharmaceutical sector.

2. Background

The enzymatic activity of Phosphodiesterases (PDE) consists of degrading cyclic nucleotides (cAMP and cGMP) through hydrolytic breakage of the 3′-phosphodiester bond, giving rise to the corresponding inactive form of 5′-monophosphate (Conti, M.; Jin, S. L., The molecular biology of cyclic nucleotide phosphodiesterases. Prog. Nucleic Acid Res. Mol. Biol. 1999, 63, 1-38). cAMP and cGMP are generated through the action of adenylate cyclase and guanylate cyclase respectively, acting as secondary messengers in the transduction of intracellular signs. One way of increasing the intracellular levels of cAMP or cGMP is through the inhibition of PDEs, since they are their only route of degradation (Vig, M.; George, A.; Sen, R.; Durdik, J.; Rath, S.; Bal, V., Commitment of activated T cells to secondary responsiveness is enhanced by signals mediated by cAMP-dependent protein kinase A-I. Mol. Pharmacol. 2002, 62, 1471-1481; Ritcher, W., 3′,5′-Cyclic nucleotide phosphodiesterases class III: Members, structure, and catalytic mechanism. Proteins 2002, 46, 278-286). The interest in developing specific PDE inhibitors is based on the antiinflammatory and immunosuppressor properties shown by agents capable of increasing the levels of intracellular cAMP (Essayan, D. M., Cyclic nucleotide phosphodiesterase (PDE) inhibitors and immunomodulation. Biochem. Pharmacol. 1999, 57, 965-973; Allison, A. C., Immunosuppressive drugs: The first 50 years and a glance forward. Immunopharmacology 2000, 47, 63-83). Therefore, selective inhibitors of cAMP-specific PDEs could be of interest as a therapy in the treatment of various diseases (Lugnier, C. Cyclic nucleotide phosphodiesterase (PDE) superfamily: A new target for the development of specific therapeutic agents. Pharmacol Ther. 2006, 109, 366-398), fundamentally alterations of the immune system, such as multiple sclerosis (Dyke, H. J.; Montana, J. G., Update on the therapeutic potential of PDE4 inhibitors. Expert Opin. Invest. Drugs 2002, 11, 1-13), inflammatory alterations (Spina, D., Phosphodiesterase-4 inhibitors in the treatment of inflammatory lung disease. Drugs 2003, 63, 2575-2594) and also disorders of the central nervous system, such as depression, cerebral ischemia damage, and Alzheimer's (Menniti, F. S.; Faraci, W. S.; Schmidt, C. J. Phosphodiesterases in the CNS: targets for drug development. Nat Rev Drug Discov. 2006, 5, 660-670).

Of the eleven described families of PDEs, those expressed in T-lymphocytes are mainly 3B, 4A, 4B, 4D and 7A1 (Ekholm, D.; Hemmer, B.; Gao, G.; Vergelli, M.; Martin, R.; Manganiello, V., Differential expression of cyclic nucleotide phosphodiesterase 3 and 4 activities in human T cell clones specific for myelin basic protein. J. Immunol. 1997, 159, 1520-1529; Erdogan, S.; Houslay, M. D., Challenge of human Jurkat T-cells with the adenylate cyclase activator forskolin elicits major changes in cAMP phosphodiesterase (PDE) expression by up-regulating PDE3 and inducing PDE4D1 and PDE4D2 splice variants as well as down-regulating a novel PDE4A splice variant. Biochem. J. 1997, 321, 165-175; Giembycz, M. A.; Corrigan, C. J.; Seybold, J.; Newton, R.; Barnes, P. J., Identification of cyclic AMP phosphodiesterases 3, 4 and 7 in human CD4+ and CD8+T-lymphocytes: Role in regulating proliferation and the biosynthesis of interleukin-2. Br. J. Pharmacol. 1996, 118, 1945-1958). Given that PDE4 is the major isoenzyme in T-cells, the development of PDE4 inhibitors has been the focus of most interest. In fact, selective inhibitors of this enzyme have demonstrated the capacity to reduce the production of inflammatory cytokines (Giembycz, M. A.; Corrigan, C. J.; Seybold, J.; Newton, R.; Barnes, P. J., Identification of cyclic AMP phosphodiesterases 3, 4 and 7 in human CD4+ and CD8+T-lymphocytes: Role in regulating proliferation and the biosynthesis of interleukin-2. Br. J. Pharmacol. 1996, 118, 1945-1958; Manning, C. D.; Burman, M.; Christensen, S. B.; Cieslinski, L. B.; Essayan, D. M.; Grous, M.; Torphy, T. J.; Barnette, M. S., Suppression of human inflammatory cell function by subtype-selective PDE4 inhibitors correlates with inhibition of PDE4A and PDE4B. Br. J. Pharmacol. 1999, 128, 1393-1398), which may have an application in the treatment of asthma, rheumatoid arthritis, and multiple sclerosis among other diseases (Houslay, M. D.; Schafer, P.; Zhang, K. Y. J. Phosphodiesterase-4 as a therapeutic target. Drug Discov. Today 2005, 10, 1503-1519). Currently, two PDE4 inhibitors, cilomilast and roflumilast, are in clinical phase III for the treatment of two diseases characterised by an immune and inflammatory response, asthma and chronic obstructive lung disease (COLD) (Chung, K. F. Phosphodiesterase inhibitors in airways disease. Eur. J. Pharmacol. 2006, 533, 110-117). However, these inhibitors demonstrate a high toxicity due to their strong emetic nature. With a view to avoiding these negative effects, various strategies have been considered, including the development of a second generation of PDE4 inhibitors with an improved pharmacokinetic profile and fewer secondary effects (Giembycz, M. Life after PDE4: overcoming adverse events with dual-specificity phosphodiesterase inhibitors. Curr. Opin. Pharm. 2005, 5, 238-244).

Another alternative is the development of inhibitors against other cAMP-selective PDEs that are expressed in immune cells. Specifically, the inhibition of PDE7 could be an approach for the treatment of diseases dependent on T-cell activation, since the potential for inhibiting the activity of Th1 cells by blocking the activity of this enzyme has been demonstrated (Li, L.; Yee, C.; Beavo, J. A., CD3- and CD28-dependent induction of PDE7 required for T cell activation. Science 1999, 283, 848-851; Nakata, A.; Ogawa, K.; Sasaki, T.; Koyama, N.; Wada, K.; Kotera, J.; Kikkawa, H.; Omori, K.; Kaminuma, O. Potential role of phosphodiesterase 7 in human T cell function: comparative effects of two phosphodiesterase inhibitors. Clin. Exp. Immunol. 2002, 128, 460-466). However, experiments based on the use of PDE7A^{-/-} knock out mice have not confirmed the role of PDE7A in the proliferation of T-cells and suggest that this enzyme could fulfill a different function in the regulation of the humoral immune response (Yang, G.; McIntyre, K. W.; Townsend, R. M.; Shen, H. H.; Pitts, W. J.; Dodd, J. H.; Nadler, S. G.; McKinnon, M.; Watson, A. J., Phosphodiesterase 7A-deficient mice have functional T cells. J. Immunol. 2003, 171, 6414-20). Therefore, the use of PDE7-selective inhibitors is fundamental for elucidating the real potential of PDE7A as a pharmacological target for the treatment of immune response alterations (Castro, A.; Jerez, M. J.; Gil, C.; Martínez, A. Cyclic nucleotide phosphodiesterases and their role in immunomodulatory responses: Advances in the development of specific phosphodiesterase inhibitors Med. Res. Rev. 2005, 25, 229-244). In this context, derivatives of benzo- and benzothienothiadiazine developed by the inventors of the present patent were the first described heterocyclic inhibitors of PDE7 (Martínez, A.; Castro, A.; Gil, C.; Miralpeix, M.; Segarra, V.; Doménech, T.; Beleta, J.; Palacios, J.; Ryder, H.; Miró, X.; Bonet, C.; Casacuberta, J.; Azorín, F.; Piña, B.; Puigdoménech, P. Benzyl derivatives of 2,1,3-benzo- and benzothieno[3,2-a]thiadiazine 2,2-dioxides: First phosphodiesterase 7 inhibitors. J. Med. Chem., 2000, 43, 683-689; Castro, A.; Abasolo, M. I.; Gil, C.; Segarra, V.; Martinez, A. CoMFA of benzyl derivatives of 2,1,3-benzo and benzothieno[3,2-a]thiadiazine 2,2-dioxides: clues for the design of phosphodiesterase 7 inhibitors. Eur. J. Med. Chem. 2001, 36, 333-338). Since then, spirotricyclic derivatives, sulphonamides, purines, and pyrimidines among others, have been described as inhibitors of PDE7 (Vergne, F.; Bernardelli, P.; Chevalier, E. PDE7 Inhibitors: Chemistry and Potential Therapeutic Utilites. Ann. Rep. Med. Chem. 2005, 40, 227-241; Giembycz, M. A.; Smith, S. J. Phosphodiesterase 7 (PDE7) as a therapeutic target. Drugs Fut. 2006, 31, 207-229).

The development of PDE7-selective inhibitors has made it possible to study the function of PDE7A in different cells and tissues. However, although the inhibition of PDE7A does not reduce the proliferation of T-cells per se, it does considerably increase the effect that PDE4 inhibitors have on increasing the levels of cAMP (Smith, S. J.; Cieslinski, L. B.; Newton, R.; Donnelly, L. E.; Fenwick, P. S.; Nicholson, A. G.; Barnes, P. J.; Barnette, M. S.; Giembycz, M. A. Discovery of BRL 50481 [3-(N,N-dimethylsulfonamide)-4-methyl-nitrobenzene], a selective inhibitor of phosphodiesterase 7: in vitro studies in human monocytes, lung macrophages, and CD8+ T-lymphocytes. Mol. Pharmacol. 2004, 66, 1679-89).

Consequently, the use of dual PDE4-PDE7 inhibitors could exercise control over determined functions of Th lymphocytes that are involved in inflammation (Hatzelmann, A.; Marx, D.; Steinhilber, W.; Altana Pharma AG. 2002. Phthalazinone derivatives useful as PDE4/7 inhibitors (WO02085906); Pitts, W. J.; Watson, A. J.; Dodd, J. H.; Bristol Myers Squibb. 2002. Dual inhibitors of PDE7 and PDE4. World patent (WO 02088079)), comprising a good strategy in the control of inflammatory or autoimmune processes, for example in multiple sclerosis (MS), and would also be less restricted by the secondary effects.

### BRIEF DESCRIPTION OF THE FIGURES

FIG. 1 is a synthesis diagram of the compounds of formula Ia as disclosed herein.

FIG. 2 is a synthesis diagram of the compounds of formula Ib as disclosed herein.

FIG. 3 is a synthesis diagram of the compounds of formula Ic as disclosed herein.

FIG. 4 is a synthesis diagram of the compounds of formula Id as disclosed herein.

FIG. 5 shows a measurement of the increase in intracellular cAMP of the derivatives TC 3.6 and TC 3.14.

### DETAILED DESCRIPTION OF THE PREFERRED EMBODIMENTS

Disclosed herein is a compound (understood as meaning a family of compounds) with a dual inhibitory activity on enzymes PDE7 and/or PDE4 (hereinafter the “disclosed compounds”), which presents the formula (I):

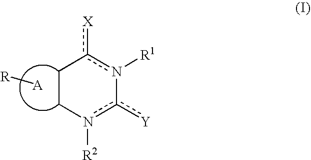

wherein:

A is a carbocycle or optionally substituted fused heterocycle of 5, 6 or 7 members, saturated or unsaturated;

- - - may be a double bond;

X and Y, are independently selected from among the group consisting of hydrogen, alkyl, ═O, ═S, —N(alkyl), —N(aryl), aryl, O-alkyl, O-aryl, S-alkyl and —S-aryl; and

R, R¹ and R², are independently selected from among the group consisting of hydrogen, halogen, alkyl, haloalkyl, aryl, cycloalkyl, (Z)ₙ-aryl, heteroaryl, —OR³; —C(O)OR³, —(Z)ₙ—C(O)OR³ and —S(O)ₜ—,

or a pharmaceutically acceptable salt, derivative, prodrug, solvate or stereoisomer thereof.

Exception: when A is benzene without substituting, X═O,Y═S
when A is benzene without substituting, X═O,Y═O,
when A is benzene without substituting, X═O,Y═S-Me
when A is thiophene without substituting, X═O,Y═S, and
when A is benzothiophene without substituting, X═O,Y═S

The present disclosure is based on the fact that the inventors have demonstrated that compounds of formula (I) are dual inhibitors of enzymes PDE7 and/or PDE4, more specifically, of enzymes PDE7A1 and/or PDE4D2 (Example 5), meaning that they can be used in the preparation of pharmaceutical compounds for the treatment of inflammatory or autoimmune processes.

Pharmaceutical compositions useful in the treatment of inflammatory and autoimmune diseases (hereinafter the “disclosed pharmaceutical compositions”), which comprise a compound, in a therapeutically effective quantity, of formula (I), or mixtures thereof, a pharmaceutically acceptable salt, derivative, prodrug, solvate or stereoisomer thereof together with a pharmaceutically acceptable carrier, adjuvant or vehicle, for administration to a patient, are also disclosed herein.

Further disclosed herein is the use of the disclosed compounds of formula (I), or its mixtures for the preparation of a medicine (hereinafter the “disclosed uses”), directed at treating inflammatory and/or autoimmune pathologies or diseases, including, but not limited to, intestinal inflammatory disease, inflammatory joint pathologies, atopic dermatitis and other inflammatory skin pathologies, neuritis, encephalitis, encephalomyelitis and inflammatory pathologies that affect the central nervous system (multiple sclerosis) or peripheral nervous system, myositis, vasculitis, systemic lupus erythematosus, infectious diseases with inflammation symptoms, host rejection of grafts, conjunctivitis, and inflammatory pathologies of the eye, ear, and mucous membranes.

One particular disclosed compound is a compound with the formula 4-oxo-2-thioxo-1,2,3,4-tetrahydroquinazoline (Compound Ia, FIG. 1), and more preferably, belonging, by way of illustration and without limitation, to the following group:
6-Bromo-3-phenyl-4-oxo-2-thioxo-1,2,3,4-tetrahydroquinazoline (TC.2.30),
6-Bromo-3-(2,6-difluorophenyl)-4-oxo-2-thioxo-1,2,3,4-tetrahydroquinazoline (TC.3.29),
6-Bromo-3-(2,3,4-trifluorophenyl)-4-oxo-2-thioxo-1,2,3,4-tetrahydroquinazoline (TC.3.30),
6-Bromo-3-(2-bromophenyl)-4-oxo-2-thioxo-1,2,3,4-tetrahydroquinazoline (TC.3.28),
3-(2,6-Difluorophenyl)-8-methyl-4-oxo-2-thioxo-1,2,3,4-tetrahydroquinazoline (TC.3.16),
3-(2,3,4-Trifluorophenyl)-8-methyl-4-oxo-2-thioxo-1,2,3,4-tetrahydroquinazoline (TC.3.17), and
3-(2-Bromophenyl)-8-methyl-4-oxo-2-thioxo-1,2,3,4-tetrahydroquinazoline (TC.3.14).

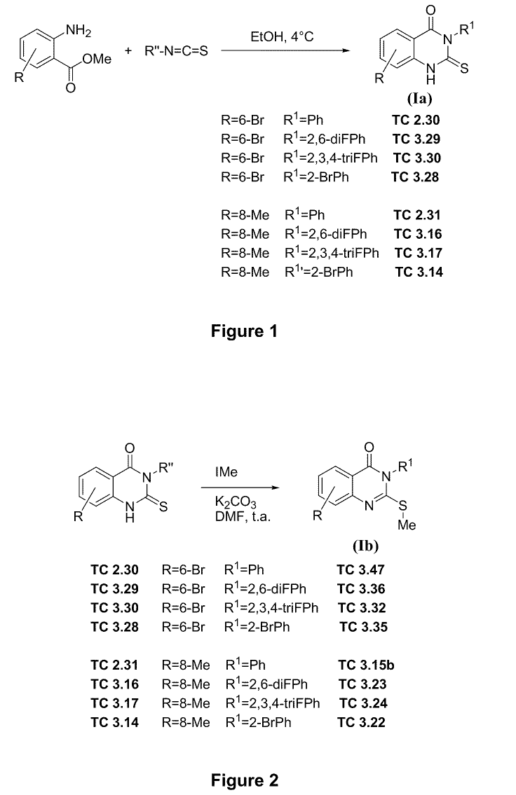

Another particular disclosed compound is a compound with the formula 2-methylthio-4-oxo-3,4-dihydroquinazoline (Compound Ib, FIG. 2), and more preferably, belonging, by way of illustration and without limitation, to the following group:
6-Bromo-3-phenyl-2-methylthio-4-oxo-3,4-dihydroquinazoline(TC.3.47),
6-Bromo-3-(2,6-difluorophenyl)-2-methylthio-4-oxo-3,4-dihydroquinazoline(TC.3.36),
6-Bromo-3-(2,3,4-trifluorophenyl)-2-methylthio-4-oxo-3,4-dihydroquinazoline(TC.3.32),
6-Bromo-3-(2-bromophenyl)-2-methylthio-4-oxo-3,4-dihydroquinazoline(TC.3.35),
3-Phenyl-8-methyl-2-methylthio-4-oxo-3,4-dihydroquinazoline(TC.3.15b),
3-(2,6-Difluorophenyl)-8-methyl-2-methylthio-4-oxo-3,4-dihydroquinazoline(TC.3.23),
3-(2,3,4-Trifluorophenyl)-8-methyl-2-methylthio-4-oxo-3,4-dihydroquinazoline(TC.3.24), and
3-(2-Bromophenyl)-8-methyl-2-methylthio-4-oxo-3,4-dihydroquinazoline(TC.3.22).

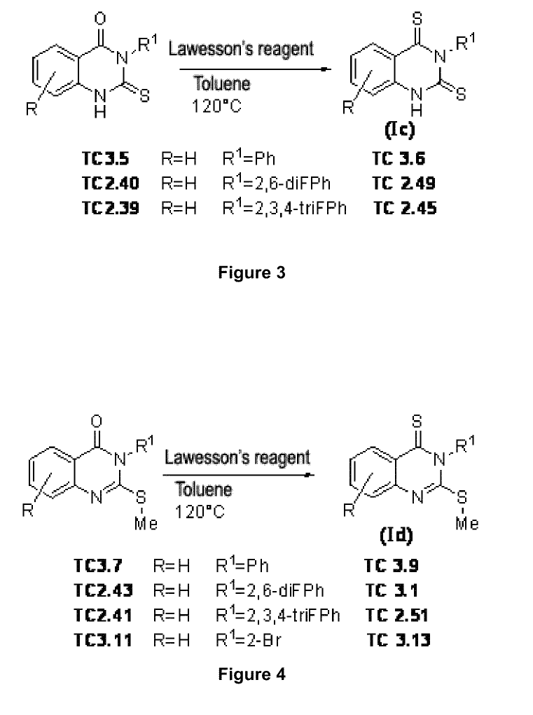

Another particular disclosed compound is a compound with the formula 2,4-dithioxo-1,2,3,4-tetrahydroquinazoline (Compound Ic, FIG. 3), and more preferably, belonging, by way of illustration and without limitation, to the following group:
3-Phenyl-2,4-dithioxo-1,2,3,4-tetrahydroquinazoline (TC.3.6),
3-(2,6-Difluorophenyl)-2,4-dithioxo-1,2,3,4-tetrahydroquinazoline (TC.2.49), and
3-(2,3,4-Trifluorophenyl)-2,4-dithioxo-1,2,3,4-tetrahydroquinazoline (TC.2.45).

Another particular disclosed compound is a compound with the formula 2-methylthio-4-thioxo-3,4-dihydroquinazoline (Compound Id, FIG. 4), and more preferably, belonging, by way of illustration and without limitation, to the following group:
3-Phenyl-2-methylthio-4-thioxo-3,4-dihydroquinazoline(TC.3.9),
3-(2,6-Difluorophenyl)-2-methylthio-4-thioxo-3,4-dihydroquinazoline(TC.3.1),
3-(2,3,4-Trifluorophenyl)-2-methylthio-4-thioxo-3,4-dihydroquinazoline(TC.2.51), and
3-(2-Bromophenyl)-2-methylthio-4-thioxo-3,4-dihydroquinazoline(TC.3.13).

The disclosed compounds represented by the formula (I), and more specifically, the compounds of formula Ia, Ib, Ic and Id, and the specific compounds belonging to these sub-families as described above may include isomers, depending on the presence of multiple bonds (for example, Z, E), including optical or enantiomeric isomers, depending on the presence of chiral centres. The isomers, enantiomers, or individual diastereoisomers and mixtures thereof fall within the scope of this disclosure. The enantiomers or individual diastereoisomers, as well as the mixtures thereof, may be separated using conventional techniques.

As referred to herein, the term “derivative” includes pharmaceutically acceptable compounds, in other words, derivatives of the compounds of the formula (I) which may be used in the preparation of a medicine, as well as pharmaceutically unacceptable derivatives since these may be useful in the preparation of pharmaceutically acceptable derivatives. The nature of the pharmaceutically acceptable derivative is not critical as long as it is pharmaceutically acceptable.

At the same time, the scope of the present disclosure includes the prodrugs of the compounds of formula (I). The term “prodrug” as referred to herein includes any compound derived from a compound of formula (I), for example, esters, including the esters of carboxylic acids, the esters of amino acids, phosphate esters, sulphonate esters of metal salts, etc., carbamates, amides, etc., which, when administered to an individual is capable of providing, directly or indirectly, said compound of formula (I) in said individual. Advantageously, such derivative is a compound that increases the bioavailability of the compound of formula (I) when administered to an individual or liberates the compound of formula (I) in a biological compartment. The nature of such derivative is not critical as long as it can be administered to an individual and provides the compound of formula (I) in a biological compartment of an individual. The preparation of such prodrug can be effected through conventional methods known to experts in the field.

The disclosed compounds may be in crystalline form as free compounds or as solvates and the intention is for both forms to be within the scope of the present disclosure. In this regard, the term “solvate”, as used herein, includes both pharmaceutically acceptable solvates, in other words, solvates of the compound of formula (I) that may be used in the preparation of a medicine, as well as pharmaceutically unacceptable solvates, which may be useful in the preparation of pharmaceutically acceptable solvates or salts. The nature of the pharmaceutically acceptable solvate is not critical as long as it is pharmaceutically acceptable. In a particular embodiment, the solvate is a hydrate. The solvates may be obtained through conventional solvation methods well known to technicians in the field.

For their application in therapy, the compounds of formula (I), their isomers, salts, prodrugs or solvates, will be, preferably, in a pharmaceutically acceptable or substantially pure form, in other words, will have a pharmaceutically acceptable level of purity excluding standard pharmaceutical additives such as solvents and carriers, and not including material considered toxic at normal dosage levels. The purity levels for the active principle are preferably more than 50%, more preferably more than 70%, more preferably more than 90%. In a preferred embodiment, they are more than 95% of the compound of formula (I), or of its salts, solvates or prodrugs.

Unless otherwise indicated, the disclosed compounds also include compounds that differ only in the presence of one or more isotopically enriched atoms. For example, compounds with said structure, excluding the substitution of one hydrogen by one deuterium or by tritium, or the substitution of one carbon for one carbon enriched in ¹³C or ¹⁴C or one nitrogen enriched in ¹⁵N, are within the scope of this disclosure.

The compounds described herein, their pharmaceutically acceptable salts, prodrugs and/or solvates as well as the pharmaceutical compositions containing them may be used in conjunction with other additional drugs in order to offer combination therapy. Said additional drugs may form part of the same pharmaceutical composition, or, alternatively, may be provided in the form of a separate composition for simultaneous or non-simultaneous administration with the pharmaceutical composition comprising a compound of formula (I), or prodrug, solvate, derivative or pharmaceutically acceptable salt thereof.

The disclosed compounds of formula (I) may be obtained or produced via chemical synthesis or obtained from a natural matter of different origin. In the present disclosure, a synthetic route is described for the disclosed compounds of formula (I) and their families (see FIGS. 1 to 4, and Examples 1 to 4), which may be carried out in practice using the data described herein by an skilled technician in the field. The syntheses for obtaining the disclosed compounds Ia, Ib, Ic and Id, as described herein, constitute another aspect of the present disclosure.

Particular disclosed pharmaceutical compositions are those wherein the compound of formula (I) belongs to one, or to several, of the following families of compounds:
4-oxo-2-thioxo-1,2,3,4-tetrahydroquinazoline (Compound Ia, FIG. 1),
2-methylthio-4-oxo-3,4-dihydroquinazoline (Compound Ib, FIG. 2),
2,4-dithioxo-1,2,3,4-tetrahydroquinazoline (Compound Ic, FIG. 3), and
2-methylthio-4-thioxo-3,4-dihydroquinazoline (Compound Id, FIG. 4),
   and, more preferably, a compound belonging, by way of illustration and without limitation, to the following group:
6-Bromo-3-phenyl-4-oxo-2-thioxo-1,2,3,4-tetrahydroquinazoline (TC.2.30),
6-Bromo-3-(2,6-difluorophenyl)-4-oxo-2-thioxo-1,2,3,4-tetrahydroquinazoline (TC.3.29),
6-Bromo-3-(2,3,4-trifluorophenyl)-4-oxo-2-thioxo-1,2,3,4-tetrahydroquinazoline (TC.3.30),
6-Bromo-3-(2-bromophenyl)-4-oxo-2-thioxo-1,2,3,4-tetrahydroquinazoline (TC.3.28),
3-(2,6-Difluorophenyl)-8-methyl-4-oxo-2-thioxo-1,2,3,4-tetrahydroquinazoline (TC.3.16),
3-(2,3,4-Trifluorophenyl)-8-methyl-4-oxo-2-thioxo-1,2,3,4-tetrahydroquinazoline (TC.3.17),
3-(2-Bromophenyl)-8-methyl-4-oxo-2-thioxo-1,2,3,4-tetrahydroquinazoline (TC.3.14),
6-Bromo-3-phenyl-2-methylthio-4-oxo-3,4-dihydroquinazoline(TC.3.47),
6-Bromo-3-(2,6-difluorophenyl)-2-methylthio-4-oxo-3,4-dihydroquinazoline(TC.3.36),
6-Bromo-3-(2,3,4-trifluorophenyl)-2-methylthio-4-oxo-3,4-dihydroquinazoline(TC.3.32),
6-Bromo-3-(2-bromophenyl)-2-methylthio-4-oxo-3,4-dihydroquinazoline(TC.3.35),
3-Phenyl-8-methyl-2-methylthio-4-oxo-3,4-dihydroquinazoline(TC.3.15b),
3-(2,6-Difluorophenyl)-8-methyl-2-methylthio-4-oxo-3,4-dihydroquinazoline(TC.3.23),
3-(2,3,4-Trifluorophenyl)-8-methyl-2-methylthio-4-oxo-3,4-dihydroquinazoline(TC.3.24),
3-(2-Bromophenyl)-8-methyl-2-methylthio-4-oxo-3,4-dihydroquinazoline(TC.3.22),
3-Phenyl-2,4-dithioxo-1,2,3,4-tetrahydroquinazoline (TC.3.6),
3-(2,6-Difluorophenyl)-2,4-dithioxo-1,2,3,4-tetrahydroquinazoline (TC.2.49),
3-(2,3,4-Trifluorophenyl)-2,4-dithioxo-1,2,3,4-tetrahydroquinazoline (TC.2.45).
3-Phenyl-2-methylthio-4-thioxo-3,4-dihydroquinazoline(TC.3.9),
3-(2,6-difluorophenyl)-2-methylthio-4-thioxo-3,4-dihydroquinazoline(TC.3.1),
3-(2,3,4-trifluorophenyl)-2-methylthio-4-thioxo-3,4-dihydroquinazoline(TC.2.51),
3-(2-bromophenyl)-2-methylthio-4-thioxo-3,4-dihydroquinazoline(TC.3.13),
   or any of their R, S enantiomeric forms and/or their racemic mixtures.

The pharmaceutically acceptable adjuvants and vehicles that may be used in said compositions are the adjuvants and vehicles known to technicians in the field and commonly used in the preparation of therapeutic compositions.

In the sense used in this description, the expression “therapeutically effective quantity” refers to the quantity of the agent or compound capable of developing inhibition of the enzymes PDE7 and PDE4, calculated to produce the required effect and, in general, will be determined, among other causes, by the inherent characteristics of the compounds, as well as age, state of the patient, severity of the alteration or disorder, including route and frequency of administration.

In another particular embodiment, said therapeutic composition is prepared in solid form or in aqueous suspension, in a pharmaceutically acceptable solvent. The therapeutic composition provided by this invention may be administered via any appropriate route of administration, and to this effect said composition will be formulated in the suitable pharmaceutical format for the selected administration route. In a particular embodiment, the therapeutic composition will be administered by oral, topical, rectal or parenteral route (including subcutaneous, intraperitoneal, intradermic, intramuscular, intravenous, etc.). A review of the various pharmaceutical way of administering medicines and the excipients required to obtain them can be found, for example, in the “Treaty of Galenic Pharmacy” “Tratado de Farmacia Galénica”, C. Faulí i Trillo, 1993, Luzán 5, S. A. Ediciones, Madrid, or in other usual or similar ones of the Spanish and US Pharmacopoeia.

The disclosed uses are compatible with their use in protocols wherein the compounds of formula (I), or mixtures thereof are used by themselves or in combination with other treatment or any other medical procedure.

Throughout the description and the claims, the word “comprise” and its variants is not intended to exclude other technical characteristics, additives, components or steps. For those skilled in the field, other objects, advantages and characteristics of the invention will become apparent from the disclosure. The following examples are provided by way of illustration and are not intended to be limiting.

### EXAMPLES

### Example 1

### General procedure for obtaining the derivatives of 4-oxo-2-thioxo-1,2,3,4-tetrahydroquinazoline (Compound Ia, FIG. 1)

To a solution of the derivative of 2-aminobenzoic acid in corresponding ethanol the isothiocyanate corresponding in each case (1 equivalent) is added at 0° C. The mixture of the reaction is agitated at 4° C., and the formation of a precipitate is observed. The solid is isolated through vacuum filtration obtaining the required product. Only in the indicated cases was purification of the solid through column chromatography necessary.

### 6-Bromo-3-phenyl-4-oxo-2-thioxo-1,2,3,4-tetrahydroquinazoline (TC.2.30)

Obtained following the general methodology described above. Reagents: 5-bromo-2-aminobenzoic acid (800 mg, 3.70 mmol), phenylisothiocyanate (0.44 mL, 3.70 mmol), ethanol (37.03 mL). Reaction time: 3 days. Product: white solid (493.70 mg, 40%).

¹H-RMN (300 MHz, DMSO-d₆), δ: 13.22 (sa, 1H, NH); 8.09 (d, 1H, J=2.1 Hz, H-5); 8.03 (dd, 1H, J=8.7 and 2.1 Hz, H-7); 7.50-7.37 (m, 4H, H-c, H-c′, H-d, H-8); 7.35 (d, 2H, J=7.3 Hz, H-b, H-b′).

¹³C-RMN (75 MHz, DMSO-d₆), δ: 176.0 (C-2); 158.7 (C-4); 139.1 (C-a); 138.8 (C-8a); 138.1 (C-7); 129.2 (C-5); 128.9 (2C, C-c, C-c′); 128.8 (2C, C-b, C-b′); 128.1 (C-d); 118.2 (C-8); 118.0 (C-4-a); 115.7 (C-6).

EM (ES): m/z=333, 335 (M+H)⁺, 355, 357 (M+Na)⁺.

Element analysis for C₁₄H₉BrN₂OS:

Calculated: C % 50.46; H % 2.72; N % 8.41; S % 9.62.

Found: C % 50.19; H % 2.88; N % 8.35; S % 9.34.

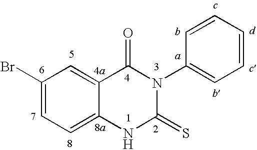

### 6-Bromo-3-(2,6-difluorophenyl)-4-oxo-2-thioxo-1,2,3,4-tetrahydroquinazoline (TC.3.29)

Obtained following the general methodology described above. Reagents: 5-bromo-2-aminobenzoic acid (600 mg, 2.77 mmol), 2,6-difluorophenylisothiocyanate (0.35 mL, 2.77 mmol), ethanol (27.70 mL). Reaction time: 17 days. Product: white solid (354.30 mg, 35%).

¹H-RMN (300 MHz, DMSO-d₆), δ: 13.56 (sa, 1H, NH); 8.07 (d, 1H, J=2.2 Hz, H-5); 8.01 (dd, 1H, J=8.7 and 2.2 Hz, H-7); 7.66-7.56 (m, 1H, H-d); 7.42 (d, 1H, J=8.7 Hz, H-8); 7.33-7.28 (m, 2H, Hc, Hc′).

¹³C-RMN (75 MHz, DMSO-d₆), δ: 174.5 (C-2); 157.7 (dd, 2C, J=248.7 and 4.4 Hz, C-b, C-b′); 157.6 (C-4); 139.2 (C-7); 138.9 (C-8a); 131.6 (t, 1C, J=10.0 Hz, C-d); 129.4 (C-5); 118.6 (C-8); 116.7 (C-6); 116.5 (C-4-a); 114.9 (t, 1C, J=16.8 Hz, C-a); 112.2 (dd, 2C, J=19.6 and 2.6 Hz, C-c, C-c′).

EM (ES): m/z=369, 371 (M+H)⁺.

Element analysis for C₁₄H₇BrF₂N₂OS:

Calculated: C % 45.55; H % 1.91; N % 7.59; S % 8.69.

Found: C % 45.31; H % 2.19; N % 7.47; S % 8.41.

HPLC: purity=98%.

### 6-Bromo-3-(2,3,4-trifluorophenyl)-4-oxo-2-thioxo-1,2,3,4-tetrahydroquinazoline (TC.3.30)

Obtained following the general methodology described above. Reagents: 5-bromo-2-aminobenzoic acid (600 mg, 2.77 mmol), 1,3,4-trifluorophenylisothiocyanate (0.36 mL, 2.77 mmol), ethanol (27.70 mL). Reaction time: 4 days. Product: white solid (540.10 mg, 50%).

¹H-RMN (300 MHz, DMSO-d₆), δ: 13.43 (sa, 1H, NH); 8.04 (d, 1H, J=2.2 Hz, H-5); 7.99 (dd, 1H, J=8.7 and 2.2 Hz, H-7); 7.56-7.42 (m, 2H, H-c, H-b); 7.39 (d, 1H, J=8.7 Hz, H-8).

¹³C-RMN (75 MHz, DMSO-d₆), δ: 175.1 (C-2); 158.2 (C-4); 150.3 (d, 1C, J=257.1 Hz, C-d); 146.5 (dd, 1C, J=246.4 and 14.4 Hz, C-b′); 139.2 (ddd, 1C, J=231.2, 15.9 and 14.1 Hz, C-c′); 138.8 (C-8a); 138.7 (C-7); 129.3 (C-5); 126.0 (dd, 1C, J=8.4 and 3.6 Hz, C-b); 123.8 (dd, 1C, J=10.7 and 4.1 Hz, C-a); 118.4 (C-8); 117.3 (C-4-a); 116.4 (C-6); 112.7 (dd, 1C, J=18.0 and 3.4 Hz, C-c).

EM (IE): m/z=366, 368 (M⁺-F, 91,100), 386, 388 (M⁺, 18, 19).

Element analysis for C₁₄H₆BrF₃N₂OS:

Calculated: C % 43.43; H % 1.56; N % 7.24; S % 8.28.

Found: C % 43.11; H % 1.68; N % 7.07; S % 7.98.

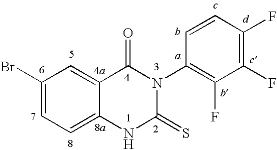

### 6-Bromo-3-(2-bromophenyl)-4-oxo-2-thioxo-1,2,3,4-tetrahydroquinazoline (TC.3.28)

Obtained following the general methodology described above. Reagents: 5-bromo-2-aminobenzoic acid (600 mg, 2.77 mmol), 2-bromophenylisothiocyanate (0.37 mL, 2.77 mmol), ethanol (27.70 mL). Reaction time: 9 days. Product: white solid (584.70 mg, 51%).

¹H-RMN (300 MHz, DMSO-d₆), δ: 13.30 (sa, 1H, NH); 8.05 (d, 1H, J=2.2 Hz, H-5); 7.98 (dd, 1H, J=8.7 and 2.2 Hz, H-7); 7.76 (d, 1H, J=7.5 Hz, H-c′) 7.54-7.46 (m, 2H, H-c, H-b); 7.42-7.35 (m, 2H, H-8, H-d).

¹³C-RMN (75 MHz, DMSO-d₆), δ: 175.0 (C-2); 158.0 (C-4); 138.8 (C-7); 138.7 (C-8a); 138.2 (C-a); 132.9 (C-c′); 131.2 (C-b); 130.4 (C-d); 129.4 (C-5); 128.7 (C-c); 122.2 (C-b′); 118.3 (C-8); 117.5 (C-4-a); 116.3 (C-6).

EM (IE): m/z=331, 333 (M⁺-Br, 100, 98).

EM (ES): m/z=413 (M+H)⁺, 825 (2M+H)⁺, 887 (2M+Na+K+H)⁺.

Element analysis for C₁₄H₈Br₂N₂OS:

Calculated: C % 40.80; H % 1.96; N % 6.80; S % 7.78.

Found: C % 40.46; H % 2.23; N % 6.77; S % 7.65.

HPLC: purity=97%.

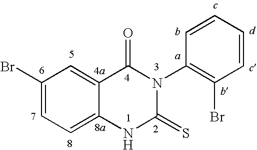

### 8-Methyl-3-phenyl-2-mercapto-4-oxo-3,4-dihydroquinazoline(TC.2.31)

Obtained following the general methodology described above. Reagents: 3-methyl-2-aminobenzoic acid (50 mg, 0.33 mmol), phenylisothiocyanate (39.57 μL, 0.33 mmol), ethanol (3.30 mL). Reaction time: 3 days. The solid obtained was used in the following step without subsequent purification.

### 3-(2,6-Difluorophenyl)-8-methyl-4-oxo-2-thioxo-1,2,3,4-tetrahydroquinazoline (TC.3.16)

Obtained following the general methodology described above. Reagents: 3-methyl-2-aminobenzoic acid (700 mg, 4.63 mmol), 2,6-difluorophenylisothiocyanate (0.59 mL, 4.63 mmol), ethanol (46.30 mL). Reaction time: 13 days. Purification: column chromatography using as eluent hexane/ethyl acetate (5:1). Product: white solid (642.70 mg, 46%).

¹H-RMN (300 MHz, CDCl₃), δ: 9.38 (sa, 1H, NH); 8.07 (d, 1H, J=7.9 Hz, H-5); 7.59 (d, 1H, J=7.3 Hz, H-7); 7.54-7.44 (m, 1H, H-d); 7.29 (dd, 1H, J=7.9 and 7.3 Hz, H-6); 7.14-7.09 (m, 2H, Hc, Hc′); 2.50 (s, 3H, Me).

¹³C-RMN (75 MHz, CDCl₃), δ: 175.5 (C-2); 158.9 (C-4); 158.4 (dd, 2C, J=251.4 and 4.2 Hz, C-b, C-b′); 137.3 (C-8a); 137.0 (C-7); 130.8 (t, 1C, J=9.75 Hz, C-d); 126.9 (C-5); 124.7 (C-6); 122.5 (C-8); 115.5 (2C, C-a, C-4-a); 112.0 (dd, 2C, J=19.8 and 3.1 Hz, C-c, C-c′); 16.2 (Me).

EM (ES): m/z=305 (M+H)⁺, 327 (M+Na)⁺, 631 (2M+Na)⁺.

EM (IE): m/z=285 (M⁺-F, 100), 304 (M⁺, 60).

Element analysis for C₁₅H₁₀F₂N₂OS:

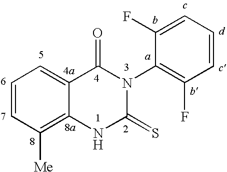

Calculated: C % 59.20; H % 3.31; N % 9.21; S % 10.54.

Found: C % 59.10; H % 3.45; N % 9.40; S % 10.65.

### 3-(2,3,4-Trifluorophenyl)-8-methyl-4-oxo-2-thioxo-1,2,3,4-tetrahydroquinazoline (TC.3.17)

Obtained following the general methodology described above. Reagents: 3-methyl-2-aminobenzoic acid (700 mg, 4.63 mmol), 2,3,4-trifluorophenylisothiocyanate (0.61 mL, 4.63 mmol), ethanol (46.30 mL). Reaction time: 4 days. Purification: column chromatography using as eluent hexane/ethyl acetate (3:1). Product: white solid (505.40 mg, 34%).

¹H-RMN (300 MHz, CDCl₃), δ: 9.36 (sa, 1H, NH); 8.01 (d, 1H, J=7.8 Hz, H-5); 7.56 (dd, 1H, J=7.4 and 0.5 Hz, H-7); 7.26 (dd, 1H, J=7.8 and 7.4 Hz, H-6); 7.16-7.04 (m, 2H, H-c, H-b); 2.46 (s, 3H, Me).

¹³C-RMN (75 MHz, CDCl₃), δ: 175.8 (C-2); 159.4 (C-4); 151.6 (ddd, 1C, J=251.0, 10.0 and 2.9 Hz, C-d); 147.6 (ddd, 1C, J=253.1, 11.0 and 3.9 Hz, C-b′); 140.8 (ddd, 1C, J=251.7, 15.8 and 13.8 Hz, C-c′); 137.2 (C-8a); 137.1 (C-7); 126.9 (C-5); 124.9 (C-6); 124.2 (dd, 1C, J=8.1 and 4.1 Hz, C-b); 123.4 (dd, 1C, J=10.9 and 4.4 Hz, C-a); 122.5 (C-8); 115.7 (C-4-a); 112.1 (dd, 1C, J=18.6 and 3.9 Hz, C-c); 16.2 (Me).

EM (IE): m/z=303 (M⁺-F, 100), 322 (M⁺, 61).

Element analysis for C₁₅H₉F₃N₂OS:

Calculated: C % 55.90; H % 2.81; N % 8.69; S % 9.95.

Found: C % 55.84; H % 3.01; N % 8.57; S % 9.97.

HPLC: purity=99%.

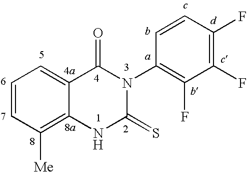

### 3-(2-Bromophenyl)-8-methyl-4-oxo-2-thioxo-1,2,3,4-tetrahydroquinazoline (TC.3.14)

Obtained following the general methodology described above. Reagents: 3-methyl-2-aminobenzoic acid (700 mg, 4.63 mmol), 2-bromophenylisothiocyanate (0.62 mL, 4.63 mmol), ethanol (46.30 mL). Reaction time: 5 days. Purification: column chromatography using as eluent hexane/ethyl acetate (8:1). Product: white solid (854.30 mg, 53%).

¹H-RMN (200 MHz, DMSO-d₆), δ: 11.98 (sa, 1H, NH); 7.86 (d, 1H, J=7.9 Hz, H-5); 7.76 (d, 1H, J=8.0 Hz, H-c′); 7.65 (d, 1H, J=7.5 Hz, H-7); 7.56-7.46 (m, 2H, H-c, H-b); 7.42-7.35 (m, 1H, H-d); 7.29 (dd, 1H, J=7.9 and 7.5 Hz, H-6); 2.53 (s, 1H, Me).

¹³C-RMN (75 MHz, DMSO-d₆), δ: 175.4 (C-2); 159.0 (C-4); 138.3 (C-8a); 138.0 (C-a); 137.2 (C-7); 132.7 (C-c′); 131.1 (C-b); 130.2 (C-d); 128.6 (C-c); 125.3 (C-5); 124.7 (C-8); 124.4 (C-6); 122.2 (C-b′); 116.0 (C-4-a); 17.4 (Me).

EM (IE): m/z=267 (M⁺-Br, 100).

Element analysis for C₁₅H₁₁BrN₂OS:

Calculated: C % 51.89; H % 3.19; N % 8.07; S % 9.23.

Found: C % 52.03; H % 3.32; N % 8.16; S % 9.16.

HPLC: purity >99%.

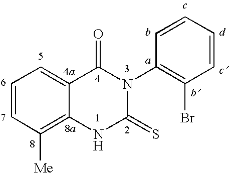

### Example 2

### General procedure for obtaining the derivatives of 2-methylthio-4-oxo-3,4-dihydroquinazoline (Compounds Ib, FIG. 2)

To a solution of the derivative of 4-oxo-2-thioxo-1,2,3,4-tetrahydroquinazoline in corresponding DMF (1 equivalent) K₂CO₃ is added (1 equivalent) and agitated at room temperature during one hour. After this time, methyl iodide is added (1.5 equivalents) and agitated at room temperature. Next, the solvent is eliminated through evaporation at reduced pressure and the solid obtained is purified as indicated in each case.

### 6-Bromo-3-phenyl-2-methylthio-4-oxo-3,4-dihydroquinazoline(TC.3.47)

Obtained following the general methodology described above. Reagents: TC.2.30 (60 mg, 0.18 mmol), K₂CO₃ (24.90 mg, 0.18 mmol), DMF (3.91 mL), methyl iodide (16.81 μL, 0.27 mmol). Reaction time: 3 hours. Purification: column chromatography using as eluent hexane/ethyl acetate (4:1). Product: white solid (60.80 mg, 97%).

¹H-RMN (300 MHz, CDCl₃), δ: 8.35 (d, 1H, J=2.2 Hz, H-5); 7.80 (dd, 1H, J=8.6 and 2.2 Hz, H-7); 7.57-7.54 (m, 3H, H-c, H-c′, H-d); 7.51 (d, 2H, J=8.6 Hz, H-8); 7.33-7.27 (m, 2H, H-b, H-b′); 2.51 (Me).

¹³C-RMN (75 MHz, CDCl₃), δ: 160.7 (C-4); 158.8 (C-2); 146.8 (C-8a); 137.7 (C-7); 135.7 (C-a); 130.9 (C-d); 129.8 (2C, C-c, C-c′); 129.7 (C-5); 129.1 (2C, C-b, C-b′); 128.1 (C-8); 121.3 (C-4-a); 119.0 (C-6); 15.5 (Me).

EM (ES): m/z=347, 349 (M+H)⁺, 717 (M+Na)⁺.

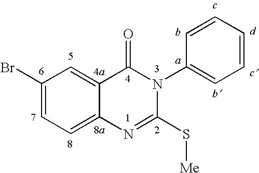

Element analysis for C₁₅H₁₁BrN₂OS:

Calculated: C % 51.89; H % 3.19; N % 8.07; S % 9.23.

Found: C % 51.71; H % 3.04; N % 7.98; S % 9.10.

### 6-Bromo-3-(2,6-difluorophenyl)-2-methylthio-4-oxo-3,4-dihydroquinazoline(TC.3.36)

Obtained following the general methodology described above. Reagents: TC.3.29 (100 mg, 0.27 mmol), K₂CO₃ (37.45 mg, 0.27 mmol), DMF (5.86 mL), methyl iodide (25.22 μL, 0.40 mmol). Reaction time: 4 hours. Purification: column chromatography using as eluent hexane/ethyl acetate (8:1). Product: white solid (102.40 mg, 99%).

¹H-RMN (300 MHz, CDCl₃), δ: 8.35 (d, 1H, J=2.3 Hz, H-5); 7.82 (dd, 1H, J=8.6 and 2.3 Hz, H-7); 7.58-7.43 (m, 1H, H-d); 7.51 (d, 1H, J=8.6 Hz, H-8); 7.14-7.08 (m, 2H, H-c, H-c′); 2.57 (Me).

¹³C-RMN (75 MHz, CDCl₃), δ: 159.4 (C-4); 158.9 (dd, 2C, J=253.7 and 3.4 Hz, C-b. C-b′); 157.8 (C-2); 146.6 (C-8a); 138.0 (C-7); 132.2 (t, 1C, J=9.8 Hz, C-d); 129.8 (C-5); 128.2 (C-8); 120.6 (C-4-a); 119.2 (C-6); 112.8 (t, 1C, J=16.9 Hz, C-a); 112.4 (dd, 2C, J=19.8 and 3.3 Hz, C-c, C-c′); 15.0 (Me).

EM (ES): m/z=383, 385 (M+H)⁺, 405, 407 (M+Na)⁺, 789 (2M+Na)⁺.

Element analysis for C₁₅H₉BrF₂N₂OS:

Calculated: C % 47.01; H % 2.37; N % 7.31; S % 8.37.

Found: C % 47.31; H % 2.37; N % 7.24; S % 8.32.

### 6-Bromo-3-(2,3,4-trifluorophenyl)-2-methylthio-4-oxo-3,4-dihydroquinazoline(TC.3.32)

Obtained following the general methodology described above. Reagents: TC.3.30 (100 mg, 0.25 mmol), K₂CO₃ (35.71 mg, 0.25 mmol), DMF (5.60 mL), methyl iodide (24.10 μL, 0.38 mmol). Reaction time: 4 hours. Purification: column chromatography using as eluent hexane/ethyl acetate (10:1). Product: white solid (66.30 mg, 64%).

¹H-RMN (300 MHz, CDCl₃), δ: 8.32 (d, 1H, J=2.3 Hz, H-5); 7.82 (dd, 1H, J=8.6 and 2.3 Hz, H-7); 7.51 (d, 1H, J=8.6 Hz, H-8); 7.21-7.06 (m, 2H, H-c, H-b); 2.56 (s, 3H, Me).

¹³C-RMN (75 MHz, CDCl₃), δ: 159.7 (C-4); 157.6 (C-2); 152.4 (ddd, 1C, J=253.3, 9.9 and 2.9 Hz, C-d); 148.0 (ddd, 1C, J=255.6, 10.9 and 4.3 Hz, C-b′); 146.5 (C-8a); 140.7 (ddd, 1C, J=253.2, 15.6 and 13.8 Hz, C-c′); 138.1 (C-7); 129.7 (C-5); 128.2 (C-8); 124.9 (dd, 1C, J=8.1 and 3.9 Hz, C-b); 120.5 (dd, 1C, J=11.1 and 3.9 Hz, C-a); 120.5 (C-4-a); 119.3 (C-6); 112.4 (dd, 1C, J=18.6 and 3.9 Hz, C-c); 15.7 (Me).

EM (ES): m/z=401, 403 (M+H)⁺, 423, 425 (M+Na)⁺.

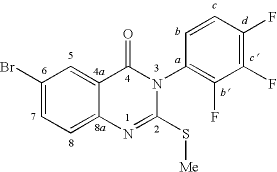

Element analysis for C₁₅H₈BrF₃N₂OS:

Calculated: C % 44.91; H % 2.01; N % 6.98; S % 7.99.

Found: C % 44.71; H % 2.02; N % 6.69; S % 7.58.

### 6-Bromo-3-(2-bromophenyl)-2-methylthio-4-oxo-3,4 dihydroquinazoline(TC.3.35)

Obtained following the general methodology described above. Reagents: TC.3.28 (125 mg, 0.30 mmol), K₂CO₃ (41.93 mg, 0.30 mmol), DMF (6.58 mL), methyl iodide (28.30 μL, 0.45 mmol). Reaction time: 5 hours. Purification: column chromatography using as eluent hexane/ethyl acetate (6:1). Product: white solid (89.10 mg, 69%).

¹H-RMN (300 MHz, CDCl₃), δ: 8.39 (d, 1H, J=2.3 Hz, H-5); 7.84 (dd, 1H, J=8.6 and 2.3 Hz, H-7); 7.80 (dd, 1H, J=7.8 and 1.3 Hz, H-c′); 7.55 (d, 1H, J=8.6 Hz, H-8); 7.56-7.50 (m, 1H, H-c); 7.47-7.39 (m, 2H, H-b, H-d); 2.57 (s, 3H, Me).

¹³C-RMN (75 MHz, CDCl₃), δ: 159.7 (C-4); 158.0 (C-2); 146.7 (C-8a); 137.8 (C-7); 135.0 (C-a); 133.9 (C-c′); 131.6 (C-b); 130.9 (C-d); 129.8 (C-5); 128.7 (C-c); 128.1 (C-8); 123.7 (C-b′); 121.0 (C-4-a); 119.0 (C-6); 15.3 (Me).

EM (ES): m/z=425, 427, 429 (M+H)⁺, 447, 449, 451 (M+Na)⁺, 875 (2M+Na)⁺.

Element analysis for C₁₅H₁₀Br₂N₂OS:

Calculated: C % 42.28; H % 2.37; N % 6.57; S % 7.52.

Found: C % 42.42; H % 2.49; N % 6.57; S % 7.23.

HPLC: purity=98%.

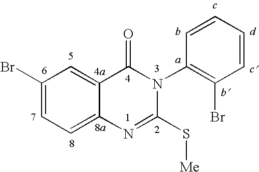

### 3-Phenyl-8-methyl-2-methylthio-4-oxo-3,4-dihydroquinazoline(TC.3.15b)

Obtained following the general methodology described above. Reagents: TC.2.31 (130 mg, 0.48 mmol), K₂CO₃ (67.04 mg, 0.48 mmol), DMF (10.54 mL), methyl iodide (45.30 μL, 0.72 mmol). Reaction time: 24 hours. Purification: column chromatography using as eluent hexane/ethyl acetate (8:1). Product: white solid (9.70 mg, 7%).

¹H-RMN (300 MHz, CDCl₃), δ: 8.09 (d, 1H, J=7.9 Hz, H-5); 7.60 (dd, 1H, J=7.2 and 0.6 Hz, H-7); 7.57-7.52 (m, 3H, H-c, H-c′, H-d); 7.35-7.27 (m, 2H, H-b, H-b′, H-6); 2.62 (s, 3H, Me); 2.54 (s, 3H, SMe).

¹³C-RMN (75 MHz, CDCl₃), δ: 162.1 (C-4); 156.6 (C-2); 146.2 (C-8a); 136.0 (C-a); 135.1 (C-7); 134.7 (C-8); 129.8 (C-d); 129.6 (2C, C-c, C-c′); 129.1 (2C, C-b, C-b′); 125.3 (C-6); 124.8 (C-5); 119.6 (C-4-a); 17.2 (Me); 15.5 (SMe).

EM (ES): m/z=283 (M+H)⁺, 305 (M+Na)⁺, 587 (2M+Na)⁺.

EM (IE): m/z=282 (M⁺, 100).

Element analysis for C₁₆H₁₄N₂OS:

Calculated: C % 68.06; H % 5.00; N % 9.92; S % 11.36.

Found: C % 67.89; H % 4.78; N % 9.94; S % 11.18.

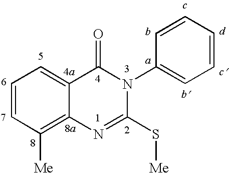

### 3-(2,6-Difluorophenyl)-8-methyl-2-methylthio-4-oxo-3,4-dihydroquinazoline(TC.3.23)

Obtained following the general methodology described above. Reagents: TC.3.16 (100 mg, 0.32 mmol), K₂CO₃ (45.46 mg, 0.32 mmol), DMF (7.13 mL), methyl iodide (30.64 μL, 0.49 mmol). Reaction time: 4 hours. Purification: column chromatography using as eluent hexane/ethyl acetate (5:1). Product: white solid (59.50 mg, 57%).

¹H-RMN (300 MHz, CDCl₃), δ: 8.10 (dd, 1H, J=7.9 and 0.7 Hz, H-5); 7.61 (dd, 1H, J=7.3 and 0.7 Hz, H-7); 7.56-7.46 (m, 1H, H-d); 7.31 (dd, 1H, J=7.9 and 7.3 Hz, H-6); 7.14-7.08 (m, 2H, H-c, H-c′); 2.62 (s, 3H, Me); 2.60 (s, 3H, SMe).

¹³C-RMN (75 MHz, CDCl₃), δ: 161.0 (C-4); 159.0 (dd, 2C, J=253.5 and 3.6 Hz, C-b, C-b′); 155.7 (C-2); 146.1 (C-8a); 135.5 (C-7); 134.9 (C-8); 131.9 (t, 1C, J=9.8 Hz, C-d); 125.5 (C-6); 124.9 (C-5); 119.1 (C-4-a); 113.1 (t, 1C, J=16.9 Hz, C-a); 112.3 (dd, 2C, J=19.9 and 3.3 Hz, C-c, C-c′); 17.1 (Me); 15.0 (SMe).

EM (IE): m/z=318 (M⁺, 100).

Element analysis for C₁₆H₁₂F₂N₂OS:

Calculated: C % 60.37; H % 3.80; N % 8.80; S % 10.07.

Found: C % 60.18; H % 3.65; N % 8.62; S % 9.78.

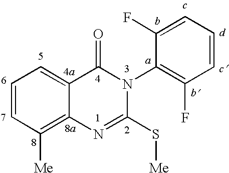

### 3-(2,3,4-Trifluorophenyl)-8-methyl-2-methylthio-4-oxo-3,4-dihydroquinazoline(TC.3.24)

Obtained following the general methodology described above. Reagents: TC.3.17 (75 mg, 0.23 mmol), K₂CO₃ (32.19 mg, 0.23 mmol), DMF (5.06 mL), methyl iodide (21.67 μL, 0.34 mmol). Reaction time: 5 hours. Purification: column chromatography using as eluent hexane/ethyl acetate (6:1). Product: white solid (43.50 mg, 56%).

¹H-RMN (300 MHz, CDCl₃), δ: 8.08 (dd, 1H, J=7.9 and 0.7 Hz, H-5); 7.61 (d, 1H, J=7.3 Hz, H-7); 7.31 (dd, 1H, J=7.9 and 7.3 Hz, H-6); 7.19-7.07 (m, 2H, H-c, H-b); 2.61 (s, 3H, Me); 2.59 (s, 3H, SMe).

¹³C-RMN (75 MHz, CDCl₃), δ: 161.3 (C-4); 155.5 (C-2); 152.3 (ddd, 1C, J=252.9, 9.9 and 2.2 Hz, C-d); 148.2 (ddd, 1C, J=254.2, 11.5 and 4.5 Hz, C-b′); 146.0 (C-8a); 140.7 (ddd, 1C, J=252.9, 15.6 and 14.1 Hz, C-c′); 135.7 (C-7); 134.9 (C-8); 125.7 (C-6); 125.0 (dd, 1C, J=8.1 and 3.9 Hz, C-b); 124.9 (C-5); 121.0 (dd, 1C, J=11.2 and 3.9 Hz, C-a); 119.1 (C-4-a); 112.3 (dd, 1C, J=18.5 and 3.8 Hz, C-c); 17.1 (Me); 15.2 (SMe).

EM (IE): m/z=336 (M⁺, 100).

Element analysis for C₁₆H₁₁F₃N₂OS:

Calculated: C % 57.14; H % 3.30; N % 8.33; S % 9.53.

Found: C % 57.35; H % 3.46; N % 8.23; S % 9.21.

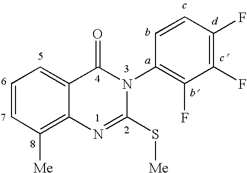

### 3-(2-Bromophenyl)-8-methyl-2-methylthio-4-oxo-3,4-dihydroquinazoline(TC.3.22)

Obtained following the general methodology described above. Reagents: TC.3.14 (250 mg, 0.72 mmol), K₂CO₃ (99.57 mg, 0.72 mmol), DMF (15.65 mL), methyl iodide (67.30 μL, 1.08 mmol). Reaction time: 6 hours. Purification: column chromatography using as eluent hexane/ethyl acetate (5:1). Product: white solid (220.40 mg, 85%).

¹H-RMN (300 MHz, CDCl₃), δ: 8.12 (d, 1H, J=7.9 Hz, H-5); 7.77 (dd, 1H, J=7.5 and 1.3 Hz, H-c′); 7.61 (d, 1H, J=7.2 Hz, H-7); 7.50 (td, 1H, J=7.5 and 1.3 Hz, H-c); 7.45-7.37 (m, 2H, H-b, H-d); 7.30 (dd, 1H, J=7.9 and 7.2 Hz, H-6); 2.64 (s, 1H, Me); 2.58 (s, 1H, SMe).

¹³C-RMN (75 MHz, CDCl₃), δ: 161.3 (C-4); 155.8 (C-2); 146.2 (C-8a); 135.4 (C-a); 135.3 (C-7); 134.7 (C-8); 133.8 (C-c′); 131.4 (C-b); 131.0 (C-d); 128.6 (C-c); 125.4 (C-6); 124.9 (C-5); 123.9 (C-b′); 119.4 (C-4-a); 17.2 (Me); 15.3 (SMe).

EM (IE): m/z=266 (M⁺-Br-Me, 47), 281 (M⁺-Br, 100), 360, 362 (M⁺, 8, 9).

Element analysis for C₁₆H₁₃BrN₂OS:

Calculated: C % 53.20; H % 3.63; N % 7.75; S % 8.88.

Found: C % 52.90; H % 3.48; N % 7.75; S % 8.59.

HPLC: purity=99%.

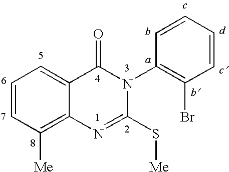

### Example 3

### General procedure for obtaining the derivatives of 2,4-dithioxo-1,2,3,4-tetrahydroquinazoline (Compounds Ic, FIG. 3)

To a solution of the derivative of 4-oxo-2-thioxo-1,2,3,4-tetrahydroquinazoline in corresponding toluene the Lawesson's reagent is added (1.5 equivalents) and is agitated to reflux. Next, the reaction mixture is allowed to cool to room temperature, and the solvent is eliminated through evaporation at reduced pressure. The solid obtained is purified as indicated in each case.

### 3-Phenyl-2,4-dithioxo-1,2,3,4-tetrahydroquinazoline (TC.3.6)

Obtained following the general methodology described above. Reagents: 3-phenyl-4-oxo-2-thioxo-1,2,3,4-tetrahydroquinazoline TC.3.5 (75 mg, 0.29 mmol), Lawesson's reagent (178.93 mg, 0.44 mmol), toluene (6.41 mL). Reaction time: 24 hours. Purification: column chromatography using as eluent hexane/ethyl acetate (9:1). Product: orange solid (50.30 mg, 63%).

¹H-RMN (300 MHz, DMSO-d₆), δ: 13.48 (sa, 1H, NH); 8.32 (dd, 1H, J=8.2 and 1.0 Hz, H-5); 7.78 (ddd, 1H, J=8.3, 7.0 and 1.0 Hz, H-7); 7.50-7.42 (m, 3H, H-c, H-c′, H-6); 7.39-7.32 (m, 2H, H-d, H-8); 7.21 (dd, 1H, J=8.3 and 1.1 Hz, H-b, H-b′).

¹³C-RMN (75 MHz, DMSO-d₆), δ: 189.8 (C-4); 172.8 (C-2); 144.2 (C-a); 135.8 (C-7); 135.2 (C-8a); 131.7 (C-5); 129.2 (2C, C-c, C-c′); 128.5 (2C, C-b, C-b′); 127.9 (C-d); 125.2 (C-8); 123.7 (C-4-a); 115.9 (C-6).

EM (ES): m/z=271 (M+H)⁺, 293 (M+Na)⁺.

EM (IE): m/z=270 (M⁺, 60).

HPLC: purity=98%.

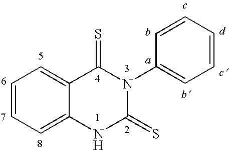

### 3-(2,6-Difluorophenyl)-2,4-dithioxo-1,2,3,4-tetrahydroquinazoline (TC.2.49)

Obtained following the general methodology described above. Reagents: 3-(2,6-difluorophenyl)-4-oxo-2-thioxo-1,2,3,4-tetrahydroquinazoline TC.2.40 (100 mg, 0.34 mmol), Lawesson's reagent (208.96 mg, 0.51 mmol), toluene (7.49 mL). Reaction time: 4 days. Purification: column chromatography using as eluent hexane/ethyl acetate (8:1). Product: orange solid (42.80 mg, 41%).

¹H-RMN (300 MHz, DMSO-d₆), δ: 13.83 (sa, 1H, NH); 8.33 (dd, 1H, J=8.2 and 1.3 Hz, H-5); 7.87 (ddd, 1H, J=8.4, 7.4 and 1.3 Hz, H-7); 7.64-7.54 (m, 1H, H-d); 7.46 (dd, 1H, J=8.4 and 0.9 Hz, H-8); 7.41 (ddd, 1H, J=8.2, 7.4 and 0.9 Hz, H-6); 7.33-7.26 (m, 2H, H-c, H-c′).

¹³C-RMN (75 MHz, DMSO-d₆), δ: 188.2 (C-4); 171.4 (C-2); 156.1 (dd, 2C, J=247.8 and 4.8 Hz, C-b, C-b′); 136.7 (C-7); 135.0 (C-8a); 131.4 (C-5); 131.1 (t, 1C, J=9.8 Hz, C-d); 125.8 (C-6); 122.3 (C-4-a); 119.7 (t, 1C, J=16.5 Hz, C-a); 116.2 (C-8); 112.2 (dd, 2C, J=19.4 and 3.0 Hz, C-c, C-c′).

EM (IE): m/z=306 (M⁺, 8).

Element analysis for C₁₄H₈F₂N₂S₂:

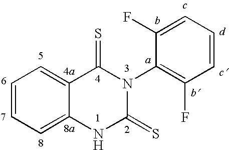

Calculated: C % 54.89; H % 2.63; N % 9.14; S % 20.93.

Found: C % 54.65; H % 2.91; N % 9.32; S % 20.78.

### 3-(2,3,4-Trifluorophenyl)-2,4-dithioxo-1,2,3,4-tetrahydrowinazoline (TC.2.45)

Obtained following the general methodology described above. Reagents: 3-(2,3,4-trifluorophenyl)-4-oxo-2-thioxo-1,2,3,4-tetrahydroquinazoline TC.2.39 (100 mg, 0.32 mmol), Lawesson's reagent (196.75 mg, 0.48 mmol), toluene (7.05 mL). Reaction time: 24 hours. Purification: column chromatography using as eluent hexane/ethyl acetate (4:1). Product: orange solid (44.00 mg, 42%).

¹H-RMN (300 MHz, DMSO-d₆), δ: 13.74 (sa, 1H, NH); 8.32 (d, 1H, J=8.2 Hz, H-5); 7.84 (ddd, 1H, J=8.2, 7.2 and 1.2 Hz, H-7); 7.56-7.37 (m, 4H, H-6, H-8, C-b, C-c).

¹³C-RMN (75 MHz, DMSO-d₆), δ: 189.4 (C-4); 172.1 (C-2); 150.3 (dd, 1C, J=247.2 and 8.1 Hz, C-d); 146.0 (dd, 1C, J=245.1 and 10.0 Hz, C-b′); 139.7 (ddd, 1C, J=246.3, 15.6 and 14.1 Hz, C-c′); 136.5 (C-7); 135.3 (C-8a); 131.6 (C-5); 128.8 (dd, 1C, J=10.2 and 3.9 Hz, C-a); 125.8 (C-8); 125.5 (dd, 1C, J=8.7 and 3.4 Hz, C-b); 123.2 (C-4-a); 116.2 (C-6); 113.1 (dd, 1C, J=18.2 and 3.4 Hz, C-c).

EM (IE): m/z=324 (M⁺, 13).

Element analysis for C₁₄H₇F₃N₂S₂:

Calculated: C % 51.84; H % 2.18; N % 8.64; S % 19.77.

Found: C % 51.67; H % 2.23; N % 8.83; S % 19.98.

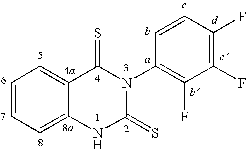

### Example 4

### General procedure for obtaining the derivatives of 2-methylthio-4-thioxo-3,4-dihydroquinazoline (Compounds Id, FIG. 4)

To a solution of the derivative of 2-methylthio-4-oxo-3,4-dihydroquinazolinecorrespondiente in toluene (1 equivalent) Lawesson's reagent is added (1.5 equivalents) and agitated to reflux. Then, the reaction mixture is allowed to cool to room temperature and the solvent is eliminated through evaporation at reduced pressure. The solid obtained is purified as indicated in each case.

### 3-Phenyl-2-methylthio-4-thioxo-3,4-dihydroquinazoline(TC.3.9)

Obtained following the general methodology described above. Reagents: 3-phenyl-2-methylthio-4-thioxo-3,4-dihydroquinazoline TC.3.7 (75 mg, 0.27 mmol), Lawesson's reagent (169.58 mg, 0.41 mmol), toluene (6.08 mL). Reaction time: 24 hours. Purification: column chromatography using as eluent hexane/ethyl acetate (6:1). Product: yellow solid (73.90 mg, 93%).

¹H-RMN (300 MHz, CDCl₃), δ: 8.68 (ddd, 1H, J=8.2, 1.3 and 0.3 Hz, H-5); 7.70 (ddd, 1H, J=8.2, 6.9 and 1.3 Hz, H-7); 7.59 (dd, 1H, J=8.2 and 0.9 Hz, H-8); 7.56-7.51 (m, 3H, H-d, H-b, H-b′); 7.37 (ddd, 1H, J=8.2, 6.9 and 0.9 Hz, H-6); 7.26-7.22 (m, 2H, H-c, H-c′); 2.49 (s, 3H, Me).

¹³C-RMN (75 MHz, CDCl₃), δ: 189.9 (C-4); 157.4 (C-2); 142.8 (C-8a); 140.4 (C-a); 134.8 (C-7); 131.1 (C-5); 129.9 (C-d); 129.8 (2C, C-b, C-b′); 128.8 (2C, C-c, C-c′); 127.5 (C-4-a); 126.9 (C-6); 126.8 (C-8); 16.0 (Me).

EM (ES): m/z=285 (M+H)⁺, 307 (M+Na)⁺, 591 (2M+Na)⁺.

Element analysis for C₁₅H₁₂N₂S₂:

Calculated: C % 63.35; H % 4.25; N % 9.85; S % 22.55.

Found: C % 63.58; H % 4.76; N % 9.83; S % 22.30.

HPLC: purity=98%.

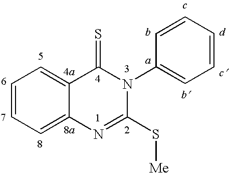

### 3-(2,6-difluorophenyl)-2-methylthio-4-thioxo-3,4-dihydroquinazoline(TC.3.1)

Obtained following the general methodology described above. Reagents: 3-(2,6-difluorophenyl)-2-methylthio-4-thioxo-3,4-dihydroquinazoline TC.2.43 (75 mg, 0.24 mmol), Lawesson's reagent (149.50 mg, 0.37 mmol), toluene (5.36 mL). Reaction time: 8 hours. Purification: column chromatography using as eluent hexane/ethyl acetate (7:1). Product: yellow solid (48.00 mg, 61%).

¹H-RMN (300 MHz, CDCl₃), δ: 8.71 (ddd, 1H, J=8.2, 1.3 and 0.5 Hz, H-5); 7.77 (ddd, 1H, J=7.9, 7.1 and 1.3 Hz, H-7); 7.64 (ddd, 1H, J=7.9, 1.1 and 0.5 Hz, H-8); 7.58-7.52 (m, 1H, H-d); 7.43 (ddd, 1H, J=8.2, 7.1 and 1.1 Hz, H-6); 7.16-7.10 (m, 2H, H-c, H-c′); 2.59 (s, 3H, Me).

¹³C-RMN (75 MHz, CDCl₃), δ: 189.2 (C-4); 158.2 (dd, 2C, J=253.7 and 4.0 Hz, C-b, C-b′); 156.6 (C-2); 142.8 (C-8a); 135.2 (C-7); 132.2 (t, 1C, J=9.7 Hz, C-d); 131.1 (C-5); 127.1 (C-6); 127.1 (C-4-a); 126.9 (C-8); 117.2 (t, 1C, J=16.5 Hz, C-a); 112.5 (dd, 2C, J=19.3 and 3.5 Hz, C-c, C-c′); 15.5 (Me).

EM (ES): m/z=321 (M+H)⁺.

Element analysis for C₁₅H₁₀F₂N₂S₂:

Calculated: C % 56.23; H % 3.15; N % 8.74; S % 20.02.

Found: C % 55.98; H % 3.56; N % 8.45; S % 19.92.

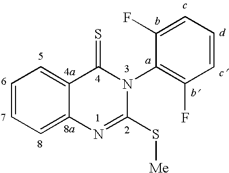

### 3-(2,3,4-trifluorophenyl)-2-methylthio-4-thioxo-3,4-dihydroquinazoline(TC.2.51)

Obtained following the general methodology described above. Reagents: 3-(2,3,4-trifluorophenyl)-2-methylthio-4-thioxo-3,4-dihydroquinazoline TC.2.41 (75 mg, 0.23 mmol), Lawesson's reagent (141.14 mg, 0.34 mmol), toluene (5.06 mL). Reaction time: 7 hours. Purification: column chromatography using as eluent hexane/ethyl acetate (6:1). Product: yellow solid (46.80 mg, 60%).

¹H-RMN (300 MHz, CDCl₃), δ: 8.68 (ddd, 1H, J=8.2, 1.3 and 0.4 Hz, H-5); 7.77 (ddd, 1H, J=8.1, 7.1 and 1.3 Hz, H-7); 7.64 (dd, 1H, J=8.1 and 0.9 Hz, H-8); 7.44 (ddd, 1H, J=8.2, 7.1 and 0.9 Hz, H-6); 7.23-7.13 (m, 1H, H-c); 7.11-7.04 (m, 1H, H-b); 2.49 (s, 3H, Me).

¹³C-RMN (75 MHz, CDCl₃), δ: 189.6 (C-4); 156.5 (C-2); 152.4 (ddd, 1C, J=253.2, 9.8 and 3.0 Hz, C-d); 147.6 (ddd, 1C, J=255.5, 10.9 and 4.1 Hz, C-b′); 142.6 (C-8a); 141.0 (ddd, 1C, J=253.0, 15.9 and 13.5 Hz, C-c′); 135.3 (C-7); 131.1 (C-5); 127.2 (C-6); 127.2 (C-4-a); 126.9 (C-8); 124.9 (dd, 1C, J=10.5 and 4.1 Hz, C-a); 124.6 (dd, 1C, J=8.2 and 4.0 Hz, C-b); 112.7 (dd, 1C, J=18.6 and 3.8 Hz, C-c); 15.8 (Me).

EM (ES): m/z=319 (M-F)⁺, 339 (M+H)⁺, 677 (2M+H)⁺.

Element analysis for C₁₅H₉F₃N₂S₂:

Calculated: C % 53.24; H % 2.68; N % 8.28; S % 18.95.

Found: C % 53.28; H % 2.73; N % 8.87; S % 18.99.

### 3-(2-bromophenyl)-2-methylthio-4-thioxo-3,4-dihydroquinazoline(TC.3.13)

Obtained following the general methodology described above. Reagents: 3-(2-bromophenyl)-2-methylthio-4-thioxo-3,4-dihydroquinazoline TC.3.11 (70 mg, 0.20 mmol), Lawesson's reagent (122.24 mg, 0.30 mmol), toluene (4.38 mL). Reaction time: 6 days. Purification: column chromatography using as eluent hexane/ethyl acetate (5:1). Product: yellow solid (32.30 mg, 45%).

¹H-RMN (300 MHz, CDCl₃), δ: 8.73 (dd, 1H, J=8.2 and 0.9 Hz, H-5); 7.79-7.73 (m, 2H, H-c′, H-7); 7.65 (d, 1H, J=8.1 Hz, H-8); 7.53 (td, 1H, J=7.5 and 1.3 Hz, H-c); 7.45-7.35 (m, 3H, H-b, H-6, H-d); 2.57 (s, 3H, Me).

¹³C-RMN (75 MHz, CDCl₃), δ: 188.8 (C-4); 156.8 (C-2); 142.8 (C-8a); 139.2 (C-a); 135.0 (C-7); 134.0 (C-c′); 131.3 (C-6); 131.1 (C-5); 130.8 (C-d); 128.9 (C-c); 127.4 (C-4-a); 127.0 (C-b); 126.9 (C-8); 123.2 (C-b′); 15.9 (Me).

EM (ES): m/z=283 (M-Br)⁺.

Element analysis for C₁₅H₁₁BrN₂S₂:

Calculated: C % 49.59; H % 3.05; N % 7.71; S % 17.65.

Found: C % 49.87; H % 3.15; N % 7.37; S % 17.25.

HPLC: purity=95%.

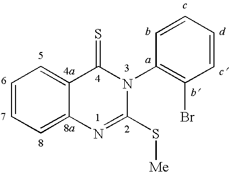

### Example 5

### Biological Studies

### PDE Inhibitor Test

The catalytic domain of PDE7A1 (aá. 130-482) and the complete enzyme of PDE4D2 (aa. 1-507) were subcloned in pET vectors, overexpressed in E. coli strain BL21, and purified to homogeneity as described in the bibliography (Wang, H., Liu, Y., Chen, Y., Robinson, H., and Ke, H. Multiple elements jointly determine inhibitor selectivity of cyclic nucleotide phosphodiesterases 4 and 7. J. Biol. Chem. 2005, 280, 30949-30955). The enzymatic activities of PDE7A1 and PDE4D2 were tested through incubation of the enzymes with 100 μL of a reaction mixture that contained 50 mM Tris.HCl (pH 8.5), 50 mM MgCl₂, 5 mM DTT, and ³H-cAMP (20000 cpm per test, Sigma-Aldrich) at room temperature during 10 minutes. The reactions were concluded by adding 200 μL 0.2 M ZnSO₄ when 20-50% of the cAMP was hydrolysed. The product of the reaction, ³H-AMP, was precipitated by adding 200 μL 0.25 M Ba(OH)₂ (Sigma-Aldrich), while the unreacted ³H-AMPc remained in the supernatant. Radioactivity of the supernatant was measured with a mixture of scintillation liquid (ScintiSafe PlusTM 50%, Fisher Scientific) using the counter LKB RackBeta 1214.

For inhibitor measurements, six concentrations of inhibitors were used in the presence of a substrate concentration equivalent to 1/10 of the K_{M} and the enzyme concentration that hydrolyses 50% of the substrate. The CI50 is defined as the compound concentration that inhibits the enzymatic activity by 50%. All experiments were carried out in duplicate.

TABLE 1
Inhibitor data of PDE7A1 and PDE4D2

| | | |
|---|---|---|
| Comp. | PDE7A1 | PDE4D2 |

| | | | |
|---|---|---|---|
| TC2.30A | >10 | μM | N.D. |
| TC3.29 | >10 | μM | N.D. |
| TC3.30 | >10 | μM | N.D. |
| TC3.28 | ~10 | μM | N.D. |
| TC3.16 | >1 | μM | N.D. |
| TC3.17 | >10 | μM | N.D. |
| TC3.14 | 1.70 ± 0.10 | μM | 34.00 ± 5.00 μM |
| TC3.47A | >1 | μM | N.D. |
| TC3.36 | 0.24 ± 0.03 | μM | 4.50 ± 0.10 μM |
| TC3.32 | 2.10 ± 0.10 | μM? | N.D. |
| TC3.35 | 1.86 ± 0.18 | μM | N.D. |
| TC3.15b | ~1 | μM | N.D. |
| TC3.23 | 0.13 ± 0.02 | μM | 1.40 ± 0.20 μM |
| TC3.24 | >1 | μM | N.D. |
| TC3.22 | 0.27 ± 0.02 | μM | 1.10 ± 0.20 μM |
| TC2.43 | 0.51 ± 0.02 | μM | 3.50 ± 0.30 μM |
| TC2.41 | ~10 | μM | N.D. |
| TC3.6 | 1.04 ± 0.08 | μM | 5.70 ± 0.03 μM |
| TC2.49 | ~1 | μM | N.D. |
| TC2.45 | >1 | μM | N.D. |
| TC3.9 | 0.84 ± 0.01 | μM | 8.00 ± 1.20 μM |
| TC3.1 | 0.1-1 | μM | N.D. |
| TC2.51 | >1 | μM | N.D. |
| TC3.13 | 0.1-1 | μM | N.D. |

### Cell Toxicity Measurements

At the same time, measurements of the cell toxicity of the compounds of formula (I) of the invention were taken.

Cell toxicity tests were carried out on a clone of Th2 lymphocytes (D10.G4.1) through flow cytometry using Propidium Iodide in order to determine cell death. This technique is based on quantifying the cells in which Propidium Iodide can penetrate because their membrane is damaged, while whole or intact cells do not allow this fluorochrome which intersperses in the nucleic acids to pass.

The cell concentration used was 1×10⁶ cells/ml, in a final volume of 200 μL, in the presence of different concentrations of the inhibitors during 48 h. After this time, the cells were washed and incubated with 5 μg/ml of Propidium Iodide during 1 min. The samples treated in this way were analysed by cytometry using BD FACSCanto™ equipment. These experiments were carried out in parallel with two commercial inhibitors: Rolipram (inhibitor of PDE4) and BRL 50481 (inhibitor of PDE7).

From the analysis of the compounds of new synthesis it can be deduced that those with the most moderate toxicity are: TC3.6, TC3.14 and TC3.15.

TABLE 2
For each inhibitor the maximum concentration at which more
than 80% cellular viability was maintained is shown in
comparison to the control of cells without inhibitor.

| Compound | Concentration (μM) |
|---|---|
| BRL50481 | >1000 |
| Rolipram | >1000 |
| TC3.6 | 1000 |
| TC3.15 | 600 |
| TC3.14 | 600 |
| TC3.9 | 100 |
| TC3.1 | 10 |
| TC3.32 | 100 |
| TC2.49 | 50 |
| TC3.23 | 10 |
| TC3.13 | 10 |
| TC3.36 | 10 |
| TC3.35 | 10 |
| TC3.22 | 5 |
| TC2.43 | 5 |

Study of Intracellular cAMP Levels

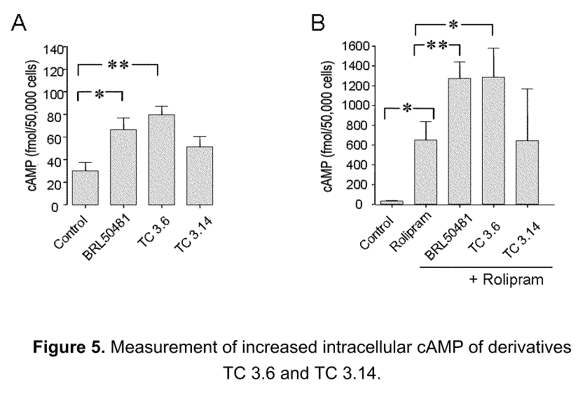

PDE inhibition has the direct effect of increasing cAMP, since their degradation is prevented. In order to verify this effect on the cell system of T-lymphocytes two compounds of formula (I) of the invention were chosen, specifically: TC 3.6 and TC 3.14 and cAMP concentration was measured using the cAMP Biotrak Enzymeimmunoassay System of Amersham Biosciences kit. As can be observed in FIG. 5, the amount of intracellular cAMP increased in a statistically significant manner with the inhibitors of PDE7 in relation to the control, although a greater increase is observed with the combination of Rolipram and the PDE7 inhibitors, with a synergy effect occurring that considerably increased the levels of detected intracellular cAMP through inhibition of PDE4.

Determination of the levels of intracellular cAMP in T-lymphocytes treated with these inhibitors has demonstrated that the inhibition of PDE7 does not produce high levels of cAMP, but is capable of synergising the effect produced by the inhibition of PDE4.

Having sufficiently described the nature of the various example embodiments, it should be stated that the aforementioned compounds and those represented in the drawings may have their details modified provided it does not alter the fundamental principle.

The invention is, of course, not limited to the examples described but cover all the variants defined in the claims. The terms “a” and “an” and “the” and similar referents used in the context of the following claims are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g. “such as”) provided herein is intended merely to better illuminate the embodiments and does not pose a limitation on the scope of the embodiments otherwise claimed. No language in the specification should be construed as indicating any non-claimed element essential to the practice of the embodiments.

The use of the term “or” in the claims is used to mean “and/or” unless explicitly indicated to refer to alternatives only or the alternatives are mutually exclusive, although the disclosure supports a definition that refers to only alternatives and “and/or.” Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments are described herein, including the best mode known to the inventors for carrying out the invention. Of course, variations on those preferred embodiments will become apparent to those of ordinary skill in the art upon reading the foregoing description. The inventors expect those of ordinary skill in the art to employ such variations as appropriate, and the inventors intend for the embodiments to be practiced otherwise than specifically described herein. Accordingly, these embodiments include all modifications and equivalents of the subject matter recited in the claims appended hereto as permitted by applicable law. Moreover, any combination of the above-described elements in all possible variations thereof are encompassed by the embodiments unless otherwise indicated herein or otherwise clearly contradicted by context.

Further, it is to be understood that the example embodiments disclosed herein are illustrative. Other modifications that may be employed are within the scope of the embodiments. Thus, by way of example, but not of limitation, alternative configurations of the present embodiments may be utilized in accordance with the teachings herein. Accordingly, the present embodiments are not limited to that precisely as shown and described in the specification and drawings.

1-7. (canceled)

8. A pharmaceutical composition for treating an inflammatory or automimmune disease comprising:
at least one of a pharmaceutically acceptable carrier, adjuvant and vehicle; and
a compound of formula (I) or a pharmaceutically acceptable salt, derivative, prodrug, solvate or stereoisomer thereof, which inhibits at least one of enzymes PDE7 and PDE4, 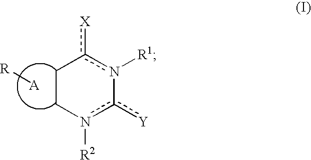
wherein A is at least one of a single bond, a double bond, a saturated or unsaturated carbocycle, and a saturated or unsaturated heterocycle having 5, 6 or 7 members,
wherein X and Y are selected independently from among the group of hydrogen, alkyl, ═O, ═S, aryl, O-alkyl, O-aryl, S-alkyl and —S-aryl,
except that when A is unsubstituted benzene and X is O, Y is one of S, O and S-Me, and when A is unsubstituted thiophene or unsubstituted benzothiophene and X is O, Y is S,
wherein R, R¹ and R² are selected independently from group of hydrogen, halogen, alkyl, haloalkyl, aryl, cycloalkyl, (CH₂)ₙ-aryl, heteroaryl, —OR³; —C(O)OR³, (CH₂)ₙ—C(O)OR³ and —S(O)ₜ—,
wherein R³ is selected independently from the group of hydrogen, halogen, alkyl, haloalkyl, aryl, cycloalkyl, (CH₂)ₙ-aryl and heteroaryl, and
wherein n is greater than or equal to 0, and t is 1 or 2.

9. The pharmaceutical composition of claim 8, wherein the compound of formula (I) is a 2-methylthio-4-thioxo-3,4-dihydroquinazoline (Compound Id).

10. The pharmaceutical composition of claim 9, wherein Compound Id is at least one of:
3-Phenyl-2-methylthio-4-thioxo-3,4-dihydroquinazoline,
3-(2,6-difluorophenyl)-2-methylthio-4-thioxo-3,4-dihydroquinazoline,
3-(2,3,4-trifluorophenyl)-2 methylthio-4-thioxo-3,4-dihydroquinazoline, and
3-(2-bromophenyl)-2-methylthio-4-thioxo-3,4-dihydroquinazoline.

11. The pharmaceutical composition of claim 8, wherein the compound of formula (I) is at least one of:
a 4-oxo-2-thioxo-1,2,3,4-tetrahydroquinazoline (Compound Ia),
a 2-methylthio-4-oxo-3,4-dihydroquinazoline (Compound Ib),
a 2,4-dithioxo-1,2,3,4-tetrahydroquinazoline (Compound Ic), and
a 2-methylthio-4-thioxo-3,4-dihydroquinazoline (Compound Id),
and any of the R or S enantiomers thereof or racemic mixtures thereof.

12. The pharmaceutical composition of claim 11, wherein the compound or mixture of compounds of formula (I) is at least one of:
6-Bromo-3-phenyl-4-oxo-2-thioxo-1,2,3,4-tetrahydroquinazoline,
6-Bromo-3-(2,6-difluorophenyl)-4-oxo-2-thioxo-1,2,3,4-tetrahydroquinazoline,
6-Bromo-3-(2,3,4-trifluorophenyl)-4-oxo-2-thioxo-1,2,3,4-tetrahydroquinazoline,
6-Bromo-3-(2-bromophenyl)-4-oxo-2-thioxo-1,2,3,4-tetrahydroquinazoline,
3-(2,6-Difluorophenyl)-8-methyl-4-oxo-2-thioxo-1,2,3,4-tetrahydroquinazoline,
3-(2,3,4-Trifluorophenyl)-8-methyl-4-oxo-2-thioxo-1,2,3,4-tetrahydroquinazoline,
3-(2-Bromophenyl)-8-methyl-4-oxo-2-thioxo-1,2,3,4-tetrahydroquinazoline,
6-Bromo-3-phenyl-2-methylthio-4-oxo-3,4-dihydroquinazoline,
6-Bromo-3-(2,6-difluorophenyl)-2-methylthio-4-oxo-3,4-dihydroquinazoline,
6-Bromo-3-(2,3,4-trifluorophenyl)-2-methylthio-4-oxo-3,4-dihydroquinazoline,
6-Bromo-3-(2-bromophenyl)-2-methylthio-4-oxo-3,4-dihydroquinazoline,
3-Phenyl-8-methyl-2-methylthio-4-oxo-3,4-dihydroquinazoline,
3-(2,6-Difluorophenyl)-8-methyl-2-methylthio-4-oxo-3,4-dihydroquinazoline,
3-(2,3,4-trifluorophenyl)-8-methyl-2-methylthio-4-oxo-3,4-dihydroquinazoline,
3-(2-bromophenyl)-8-methyl-2-methylthio-4-oxo-3,4-dihydroquinazoline,
3-Phenyl-2,4-dithioxo-1,2,3,4-tetrahydroquinazoline,
3-(2,6-Difluorophenyl)-2,4-dithioxo-1,2,3,4-tetrahydroquinazoline,
3-(2,3,4-Trifluorophenyl)-2,4-dithioxo-1,2,3,4-tetrahydroquinazoline.
3-Phenyl-2-methylthio-4-thioxo-3,4-dihydroquinazoline,
3-(2,6-difluorophenyl)-2-methylthio-4-thioxo-3,4-dihydroquinazoline,
3-(2,3,4-trifluorophenyl)-2 methylthio-4-thioxo-3,4-dihydroquinazoline, and
3-(2-bromophenyl)-2-methylthio-4-thioxo-3,4-dihydroquinazoline.

13. The pharmaceutical composition of claim 8, wherein the inflammatory or autoimmune disease is at least one of intestinal inflammatory disease, inflammatory joint pathologies, atopic dermatitis and other inflammatory skin pathologies, neuritis, encephalitis, encephalomyelitis and inflammatory pathologies affecting the central nervous system (multiple sclerosis) or peripheral nervous system, myositis, vasculitis, systemic lupus erythematosus, asthma, chronic obstructive lung disease, infectious diseases with inflammation symptoms, reactions of host rejection against grafts, conjunctivitis, and inflammatory pathologies of the eyes, ears and mucous membranes.
